(19) United States
(12) Patent Application Publication
(10) Pub. No.: US 2016/0278800 A1
(43) Pub. Date: Sep. 29, 2016

(54) TREATMENT TOOL FOR ENDOSCOPIC SURGERY

(71) Applicant: FUTAKU PRECISION MACHINERY INDUSTRY COMPANY, Kyoto-shi, Kyoto (JP)

(72) Inventors: Tetsuo HAYAMI, Kyoto-shi, Kyoto (JP); Satoru FUNAKOSHI, Kyoto-shi, Kyoto (JP); Eiji KANEHIRA, Soka-shi, Saitama (JP)

(21) Appl. No.: 14/770,986

(22) PCT Filed: Mar. 17, 2014
(86) PCT No.: PCT/JP2014/057210

§ 371 Date: Jun. 7, 2016

(30) Foreign Application Priority Data
Sep. 3, 2013 (JP) 2013-182053

(51) Int. Cl. A61B 17/29

(52) U.S. Cl. CPC A61B 17/29; A61B 2017/2903; A61B 2017/2905; A61B 2017/00309

(57) ABSTRACT

To provide a treatment tool for endoscopic surgery which has, in an insertion part, two or more bending parts that can be independently operated to bend, and which has distal forceps pieces that can be opened and closed, and rotated.

A treatment tool for endoscopic surgery comprising an insertion part provided on one end of a pipe-shaped main body and an operation part provided on another end, wherein: the insertion part has two or more bendable parts and a pair of forceps pieces; the operation part has a bending operation part that correspond to each of the bendable parts, an open and close operation part, and a rotation operation part; the bendable parts can be operated to bend by operating a first wire member connected to the bending operation part to move back and forth inside the main body in an axial direction; the forceps pieces can be operated to open and close by operating a second wire member connected to the open and close operation part to move back and forth inside the main body in an axial direction; and the forceps pieces can be operated to rotate by operating a third wire member connected to the rotation operation part to rotate about the axis inside the main body.

### TECHNICAL FIELD

The present invention relates to a treatment tool for an endoscopic surgery.

### BACKGROUND ART

An endoscopic surgery is a surgery in which a few cm holes are opened in the abdomen of a patient and cylindrical surgical tools called “trocar” are inserted through the holes and surgical operations are performed by using the treatment tools, such as endoscopes, inserted into the trocars. The surgery has an advantage of not only less physical burden for a patient but also a faster recovery, compared to incision surgeries.
Here, the treatment tool used for an endoscopic surgery can be inserted into the trocar and shall be used in the state of being inserted into the trocar. Thus, the treatment tool is subjected to severe structural constraints due to the shape of the trocar (general length: 100 mm, the inside diameter: 5 mm). Therefore the tool is difficult to use with sufficient operability. A treatment tool is known, which has a single bendable portion in the insertion part to be inserted into a patient body and also has openable/closable rotatable forceps pieces at the distal end (Patent Literature 1)

### CITATION LIST

### Patent Literature

### Patent Literature 1: Japanese Patent Laid-Open No. 2008-220972

### SUMMARY OF INVENTION

### Technical Problem

However due to only one bendable portion in the insertion part in the treatment tool, it is difficult to adjust the angle of the forceps pieces to the organ of a patient and the distance from the entrance of the trocar to the forceps pieces. Therefore, there is a risk injuring the other internal organs if an operator fails to grip them appropriately, or a plurality of treatment tools are required according to the patient physique etc.
Therefore, it is an object of the present invention to provide a treatment tool for an endoscopic surgery with excellent operability. More particularly, the present invention provides a treatment tool for an endoscopic surgery which has two or more bendable portions that can be operated to bend independently in the insertion part to be inserted into a patient body, and of which distal end forceps pieces are openable/closable and rotatable.

### Solution to Problem

A treatment tool for an endoscopic surgery according to the present invention comprises:
an insertion part disposed at one end of a pipe-like main body; and an operation part disposed at the other end of the main body,
wherein the insertion part has two or more bendable portions and a pair of forceps pieces,
the operation part has each bending operation part corresponding to the each bendable portion respectively, an open-close operation part and a rotation operation part, the bendable portions can be operated to bend by operating first linear members connected to the bending operation parts to move back and forth in the axial direction inside the main body,
the forceps pieces can be operated to open and close by operating a second linear member connected to the open-close operation part to move back and forth in the axial direction inside the main body, and
the forceps pieces can be operated to rotate by operating a third liner member connected to the rotation operation part to rotate around the axis inside the main body.
In such a configuration, the operability of the treatment tool is improved, because the treatment tool has two or more bendable portions that can be operated to bend independently in the insertion part to be inserted into a patient body and its distal end forceps pieces are openable/closable and rotatable,
In addition, the “liner member” is not limited to liner members, such as a wire, a cable or a rope, but also includes cylindrical members, such as pipe, bar-like members, such as a rod or a shaft, and any parts formed by connecting them.
Also in the configuration above,
the third liner member may be a pipe having one spirally formed slit at least in the bendable portions.
Such a configuration allows the one spiral slit in the pipe (third liner member) to improve flexibility of the pipe.
Also in the configuration above,
the third liner member may be a pipe having a plurality of spirally formed parallel slits at least in the bendable portions.
Such a configuration allows the plurality lines of spirally formed parallel slits in the pipe (third liner member) to improve flexibility of the pipe furthermore.
Also in the configuration above,
each slit may have at least one non-slit portion respectively.
Such a configuration allows the non-slit portions in the spirally formed slits in the pipe (third liner member) to reduce a spring property of the pipe around the axis of the pipe due to the formation of the spirally formed slits, to reduce the absorption of the rotational force generated by the rotation operation part, and also reduce the repulsive force from the pipe. Thus the flexibility of the pipe is secured and as a result an operator obtains a direct operation feeling of rotating the forceps pieces and can easily adjust the angle of the forceps pieces along the rotational direction thereof.
In addition, “non-slit portion” refers to discontinuous portion in the slit.
Also in the configuration above,
the third liner member may be a pipe which has a plurality of slits formed parallel to the axial direction in at least a part of an outer circumference circle vertical to the axis of the pipe, at least in the bendable portions, instead of the spiral slits above.
Such a configuration allows the plurality of slits formed parallel to the axial direction in at least a part of an outer circumference circle vertical to the axis to improve flexibility of the pipe (third liner member) without deteriorating strength against a rotational torque around the axis thereof, compared to the pipe having spiral slits.
Also in the configuration above,
the third liner member may be made of a β titanium alloy.
In such a configuration, it is possible to transmit the rotational torque to the distal end forceps pieces appropriately by using the β titanium alloy that is bendable flexibly and has excellent strength, even if a plurality of the portions are bent in the insertion part. Also it leads to reduction in the diameter of the third liner member. As described above, since the diameter of the pipe-like main body is constrained to the inside diameter of the trocar, the reduction in the diameter of the third liner member enables the plurality of the first liner members to be inserted into the main body. Therefore, the plurality of bendable portions that can be operated to bend independently in the insertion part are provided, and as a result, the operability of the treatment tool is improved.

### Advantageous Effects of Invention

As described above, since the treatment tool for an endoscopic surgery has two or more bendable portions that can be operated to bend independently, in the insertion part to be inserted into a patient body, and its distal end forceps pieces are openable/closable and rotatable, as a result, the operability of the treatment tool is improved.

### BRIEF DESCRIPTION OF DRAWINGS

FIG. 1 is a view illustrating a structure of a treatment tool for an endoscopic surgery according to an embodiment of the present invention.

FIG. 2 is an enlarged view of an insertion part in the treatment tool according to an embodiment of the present invention.

FIG. 3 is an enlarged view of a part of an operation part in the treatment tool according to an embodiment of the present invention.

FIG. 4 is a view illustrating open-close operation of forceps pieces according to an embodiment of the present invention.

FIG. 5 is a view illustrating a structure of a third liner member according to an embodiment of the present invention.

FIGS. 6 (a), (b), (d) and (d) are development views of the third liner member according to an embodiment of the present invention.

FIG. 7 is a cross-section view of the third liner member according to an embodiment of the present invention.

FIGS. 8 (a), (b-1), (b-2) and (c) are views illustrating possible forms of the treatment tool for an endoscopic surgery according to an embodiment of the present invention.

FIGS. 9 (a), (b) and (c) are views illustrating variations of a second liner member and the third liner member according to an embodiment of the present invention.

### DESCRIPTION OF EMBODIMENTS

Hereinafter, embodiments of the present invention will be described with reference to the drawings. However, the following embodiments are merely examples, and the interpretation should not be limited with the descriptions of these embodiments. Here, the same or corresponding parts are denoted by the same reference character in the drawings.

### Examples

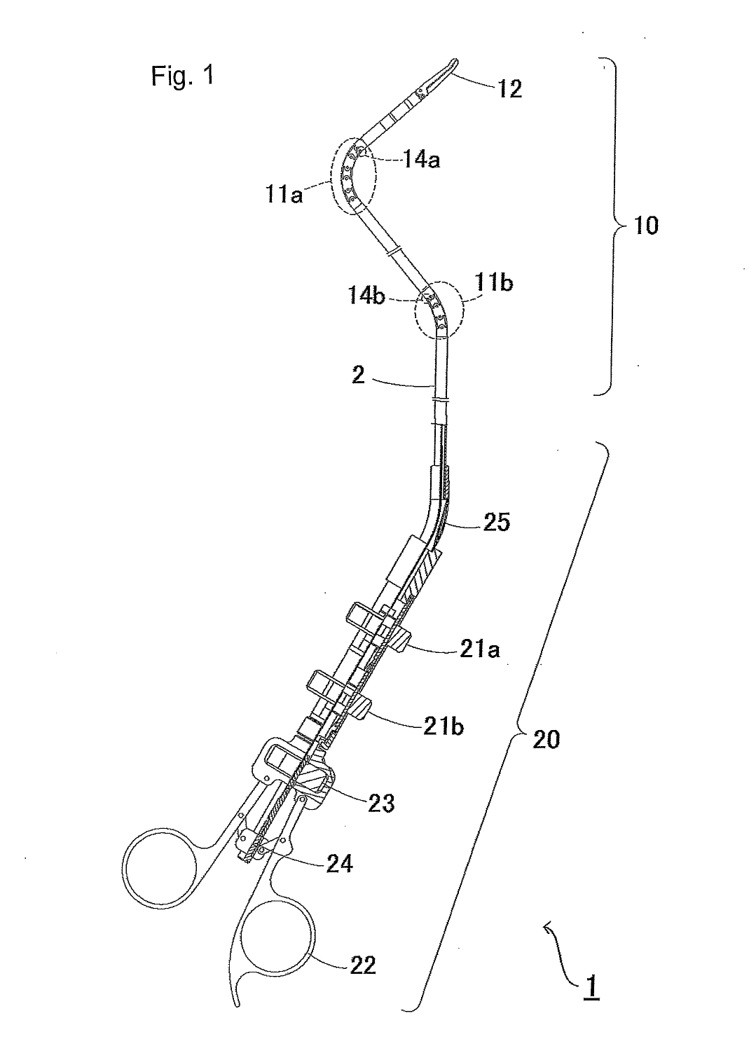

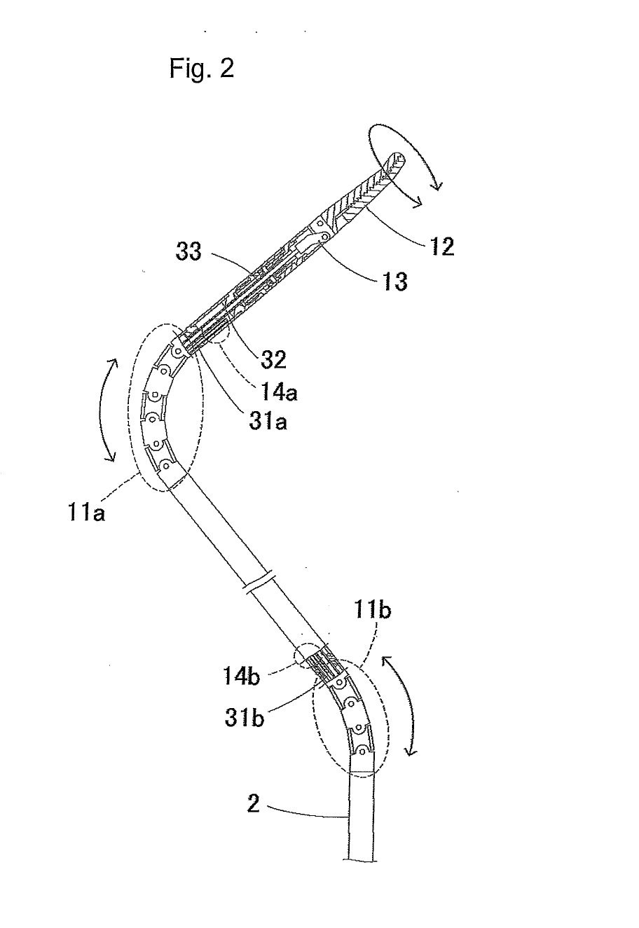

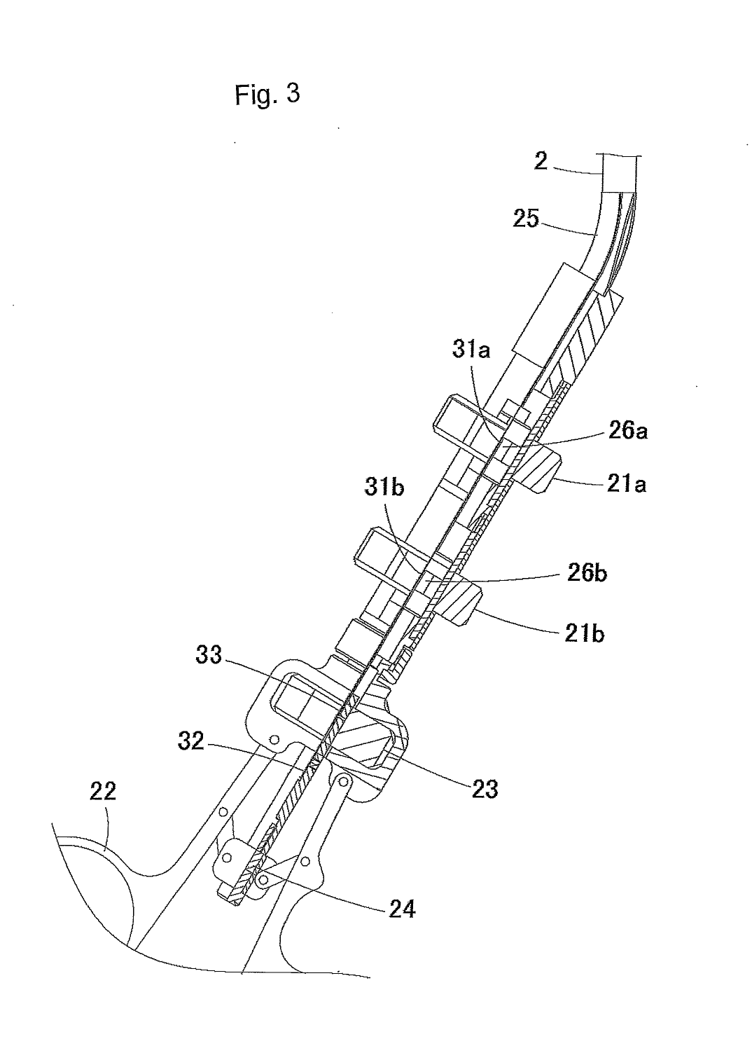

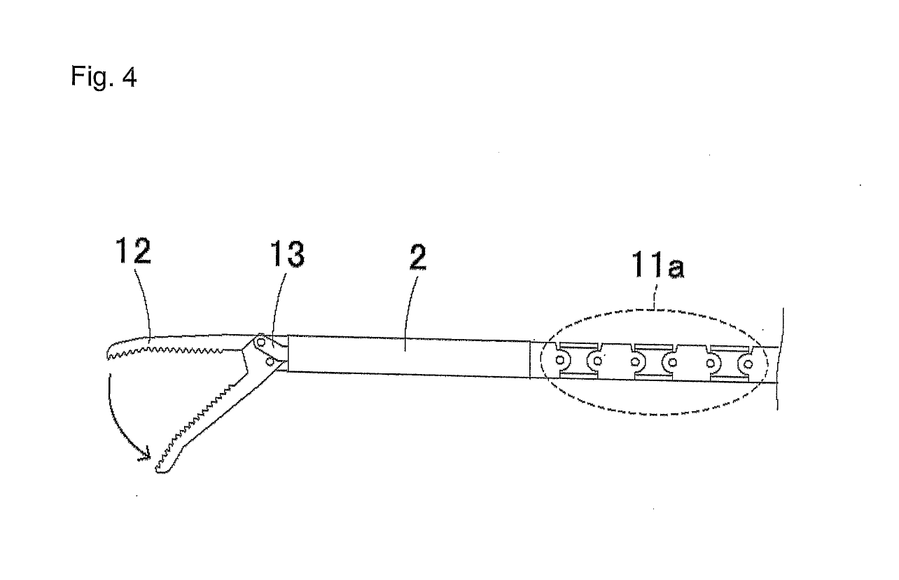

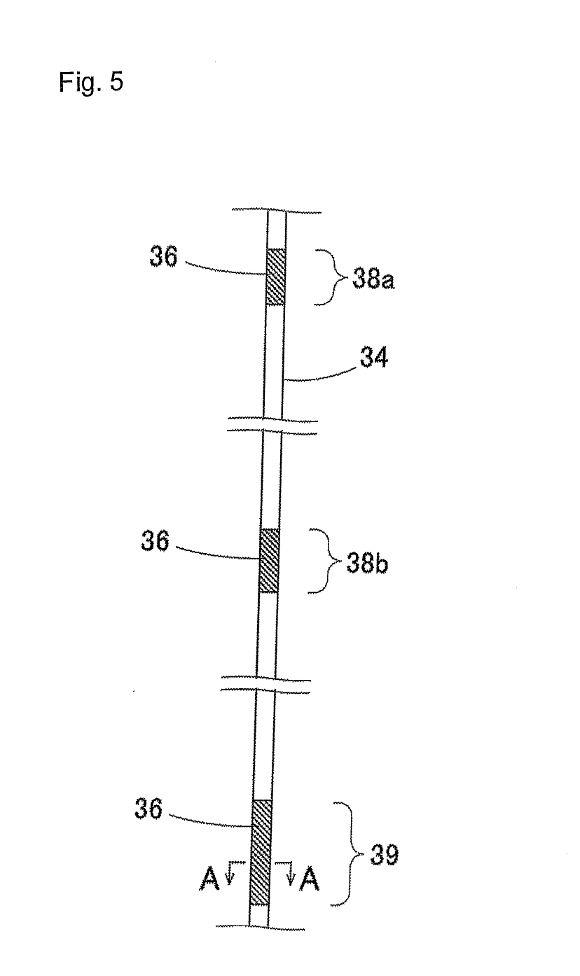

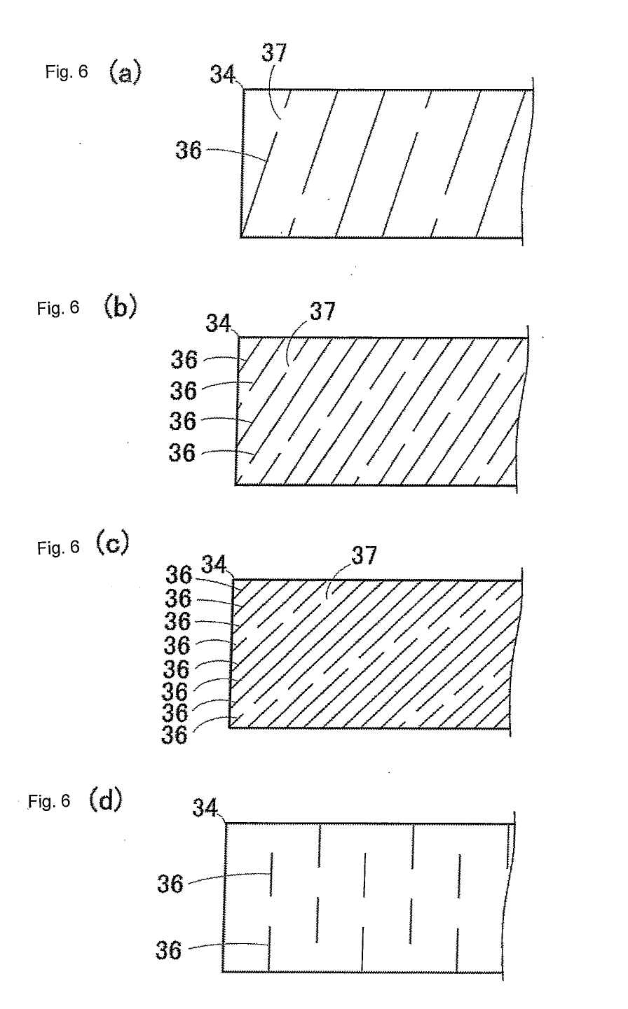

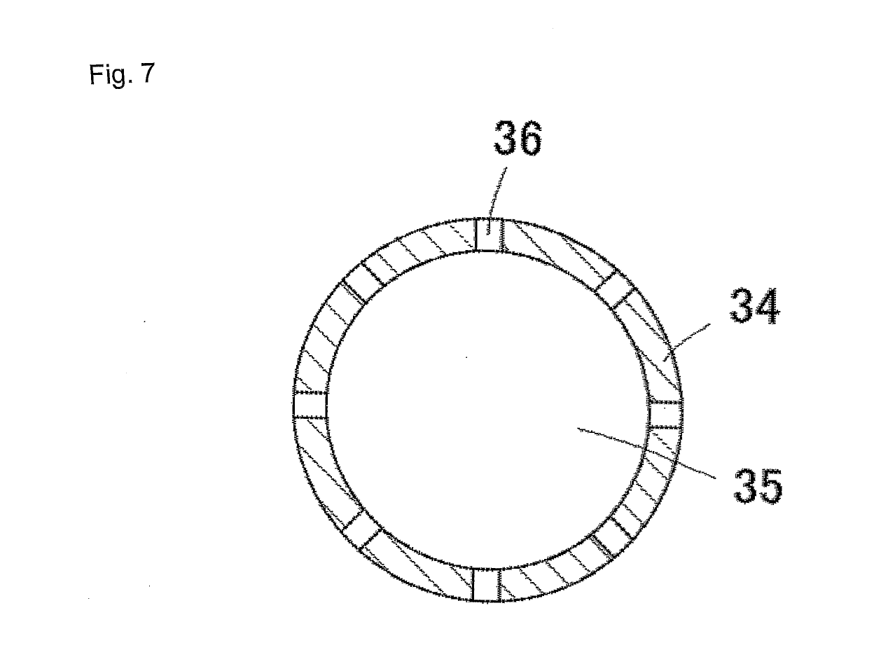

FIG. 1 is a view illustrating a structure of a treatment tool for an endoscopic surgery according to an embodiment. A treatment tool 1 includes an insertion part 10 and an operation part 20. The insertion part 10 is disposed at one end of a pipe-like main body 2, and includes two or more bendable portions 11 (a first bendable portion 11a and a second bendable portion 11b in the embodiment) and a pair of forceps pieces 12. The operation part 20 is disposed at the other end of the pipe-like main body 2, and includes each bending operation part 21 corresponding to each bendable portion 11 in the insertion part 10 (a first bending operation part 21a corresponding to the first bendable portion 11a and a second bending operation part 21b corresponding to the second bendable portion 11b), an open-close operation part 22 and a rotation operation part 23.
The operation part is provided with a bending portion 25 curved at an angle about 30 degrees to prevent other treatment tools from interfering with the treatment tool for an endoscopic surgery during surgery. The bending angle of the bending portion 25 can be appropriately changed without departing from the scope of the present invention as a matter of course, and also the bending portion 25 may be formed in a linear form. In addition, the bending portion 25 may be in a fixed form which is not deformed even if external force is applied to bending portion 25 during surgery, or may be in a variable form so that the form can be appropriately changed during surgery.
FIG. 2 is an enlarged view of an insertion part 10 of the treatment tool and FIG. 3 is an enlarged view of a part of an operation part 20 of the treatment tool, according to the embodiment. The first liner member 31a connects the first bendable portion 11a in the insertion part 10 to the first bending operation part 21 in the operation part 20. The first bendable portion 11a is operated through the first liner member 31a by the first bending operation part 21a and bends in only one direction.
The first liner member 31a is locked by a locking member 14a located on the inward side of bending in the first bendable portion 11a and on the side of the insertion part 10, and by a locking member 26a moving in conjunction with the first bending operation part 21a in the operation part 20. When the first bending operation part 21a (first bending operation dial in the embodiment) is rotated, the locking member 26a connected to the first liner member 31a will move forward and backward inside the main body axially. Thereby tensile force is applied to the locking member 14a in the first bendable portion 11a connected to the other end of the first liner member 31a so that the first bendable portion 11a is bend-driven. When the first bending operation dial 21a is set back, the bendable portion 11a returns to a straight form by a restoring force of liner members 32, 32 and 33 which are inserted into the main body 2.
The same principle enables the second bendable portion 11b to be operated through the first liner member 31b by the second bending operation portion 21b.
The first liner member 31 may be made of a stainless steel rope because materials, such as a stainless steel rope, have enough tensile strength and flexibility necessary to be bend-driven.
Unless otherwise specifically noted according to the embodiment, the bendable portion 11 represents both of the first bendable portion 11a and the second bendable portion 11b, the bending operation part 21 represents both of the first bending operation part 21a and the second bending operation part 21b, and the first liner member 31 represent both of the first liner member 31a and the first liner member 31b.
A pair of the forceps pieces 12 are arranged at the tip of the pipe-like main body 2 openably and closably. The second liner member 32, which is inserted into the main body 2, connects the forceps pieces 12 to the open-close operation part 22 through link mechanisms 13 and 24. An open-close action by open-close operation handle (open-close operation part 22) enables the second liner member 32 to move forward and backward inside the main body 2 so that the forceps pieces 12 is open-close-driven.
FIG. 4 is an enlarged view illustrating an open-close operation of a pair of the forceps pieces 12 according to an embodiment. As shown in the same figure, one of the forceps pieces 12 may be fixed and only another may be movable, or both of the forceps pieces 12 are operated together to open or close.
The second liner member 32 may be made of e.g. a wire or a rod. Examples of the material thereof are a stainless steel, a titanium or a titanium alloy. Preferably, a rod made of a nickel-titanium alloy should be used because it has excellent restoration force.
Further, a pair of the forceps pieces 12 are arranged at the tip of the pipe-like main body 2 rotatably around the axis of the pipe. The third liner member 33, which is inserted into the main body 2, connects the forceps pieces 12 to the rotation operation part 23. A rotating action of rotation operation dial (rotation operation part 23) enables the third liner member 33 to rotate around the axis thereof inside the main body 2 so that the forceps pieces 12 is rotate-driven.
The third liner member 33 may be made of a flexible material, such as a rod, a pipe or a wire. While the material may be selected depending on the size or strength required, preferably, a flexible metal, such as a nickel titanium alloy or a 6 titanium alloy, should be used. A pipe made of a β titanium alloy should be especially preferably used among them, because the pipe made of a β titanium alloy has sufficient flexibility and can transmit enough rotation operation force applied in the rotation operation part 23 to the distal end forceps pieces 12, and the second liner member 32 can be inserted into the pipe.
Since in a conventional general treatment tool for an endoscopic surgery, the rotation operation part 23 in the operation part is arranged far from the distal end forceps pieces 12 and the rotation operation force applied in the rotation operation part 23 is absorbed in the main body 2 due to the (frictional) resistance inside, enough driving force cannot be transmitted to the distal end forceps pieces 12. In addition, when a repulsive force is generated in the rotation operation part 23 and a twisting force is increased up to a specific level, the force transmitted at once to the forceps pieces 12 causes excessive rotation of the forceps pieces 12 and thereby an operator cannot obtain direct operation feeling. Especially, the more bendable portions 11 the main body 2 has, the more troubles it causes. Thus, if the distal end forceps pieces 12 are configured to be openable/closable and rotatable, the conventional treatment tool has only one bendable portion 11 as an upper limit in the insertion part 10.
In that aspect, the present embodiment uses a 6 titanium alloy, which is bendable flexibly and has excellent strength, as the liner member 33 for the rotation operation of the forceps pieces 12. Thereby this enables to transmit enough driving force to the distal end forceps pieces 12 even if a plurality of bendable portions are being bent in the insertion part 10 and also enables miniaturizing the liner member 33 in the treatment tool compared to conventional treatment tools. As described above, since the outer diameter of the pipe-like main body 2 is constrained to the inside diameter of the trocar, reduction in the diameter of the third liner member 33 enables the plurality of the first liner members 31 to be inserted into the main body 2. Therefore, the plurality of bendable portions 11 in the insertion part 10 are provided and as a result, the operability of the treatment tool is improved.
FIG. 5 is a view illustrating a structure of a third liner member 33 of the embodiment. The third liner member 33 is a pipe 34 having slits 36, in at least a first slit forming part 38a corresponding to the first bendable portion 11a and a second slit forming part 38b corresponding to the second bendable portion 11b, and the present embodiment has slits 36 even in a third slit forming part 39 corresponding to the bending portion 25. The slits described above improve the flexibility of the pipe 34.
FIGS. 6 (a), (b), (d) and (d) are development views of the third liner member 33 of the embodiment, and show forms of the slits 36 formed in the pipe 34 (third liner member 33). Note that these figs. have been changed as appropriate scale to facilitate understanding of the forms of the slits 36, the aspect ratio of the drawings are not necessarily accurate.
The third liner member 33 (pipe 34) illustrated in FIG. 6 (a) has one spirally formed slit 36 in at least the bendable portions 11. This slit 36 improves the flexibility of the pipe 34. Further the slit 36 has at least one non-slit portion 37. The non-slit portion 37 enables to reduce the spring property around the axis of the pipe 34 generated by forming the spiral slit 36, and also reduce the repulsive force of the forceps pieces 12 generated in the rotation operation. Therefore, an operator obtains direct operation feeling when rotating the forceps pieces and can easily adjust the angle of the forceps pieces along the rotational direction thereof.
The third liner member 33 (pipe 34) illustrated in FIG. 6 (b) has four spirally formed parallel slits 36 in at least the bendable portions 11. These four slits 36 improves the flexibility of the pipe 34 compared to the pipe 34 having one slit. In addition, the each four slits 36 have at least one non-slit portion 37 respectively, as well as the slit 36 having one slit.
The third liner member 33 (pipe 34) illustrated in FIG. 6 (c) has eight spirally formed parallel slits 36 in at least the bendable portions 11. The four slits 36 further improve the flexibility of the pipe 34 compared to the pipe 34 having one or four slits. However, the more slits 36 the pipe 34 has, the less strength the pipe 34 has. Thus it is not preferable to excessively increase the number of the slits. In addition, the each eight slits 36 have at least one non-slit portion 37 respectively, as well as the slit 36 having one or four slits.
The widths of the slits 36, interval between the slits 36 and the length and the number of the non-slit portions 37 are appropriately selected depending on the flexibility necessary for the pipe 34 and the rotation operation force applied by the rotation operation part 23.
FIG. 7 is a cross-section view of the third liner member 33 (pipe 34) viewed from the line A-A direction in FIG. 5. The pipe 34 having a hollow portion 35 inside include eight spirally formed parallel slits 36 and the each slit 36 has such a depth as to reach the hollow portion 35. Meanwhile, the each slit 36 may be formed only on the surface layer of the outer diameter face of the pipe 34, and is not required to reach the hollow portion 35.
The third liner member 33 (pipe 34) illustrated in FIG. 6 (d) has the plurality of slits 36 formed in parallel to the axial direction, and the slits are formed in at least a part of the outer circumference circle vertical to the axis of the pipe. The pipe 34 has the slits 36 above improves the flexibility of the pipe 34 without deteriorating the strength against a rotational torque around the axis thereof, compared to the pipe 34 having spiral slits 36. However, the pipe 34 having spiral slits 36 is superior in the aspect of the reduction of the spring property against the rotational torque around the axis thereof and the durability against the bending. In addition, the slits may be displaced from each other so that the evenly spaced slits 36 are formed around the axis thereof.
Preferably, the forms of the slits 36 formed in the pipe 34 should be selected from or combined with the examples of the forms described above by the diameter or the material of the pipe 34, or the number or the bending angles of the bendable portions 11.
Although, those described the method for improving a flexibility of the pipe 34 when the pipe 34 is used as the third liner member 33, the flexibility of a rod is improved when a rod is used as the third liner member 33, by thinning the portion around the bendable portions 11.
Although the present embodiment uses the bending operation dial as the bending operation part 21, uses the open-close operation handle as the open-close operation part 22, and uses the rotation operation dial as the rotation operation part 23, also known operation units, such as a dial, a handle or a lever, may be used.

### Modification

As described above, according to the treatment tool for an endoscopic surgery, reliable transmission of a rotation operation force applied in the rotation operation part 23 to the distal end forceps pieces 12 is one of the issues. Therefore while the present embodiment uses a pipe-like member, preferably a pipe made of a 6 titanium alloy which is bendable flexibly and has excellent strength, for the third liner member 33, the following method can also improve the transmitting performance of the rotation operation force. In addition, the following modification uses a pipe as the third liner member 33.

### Modification1

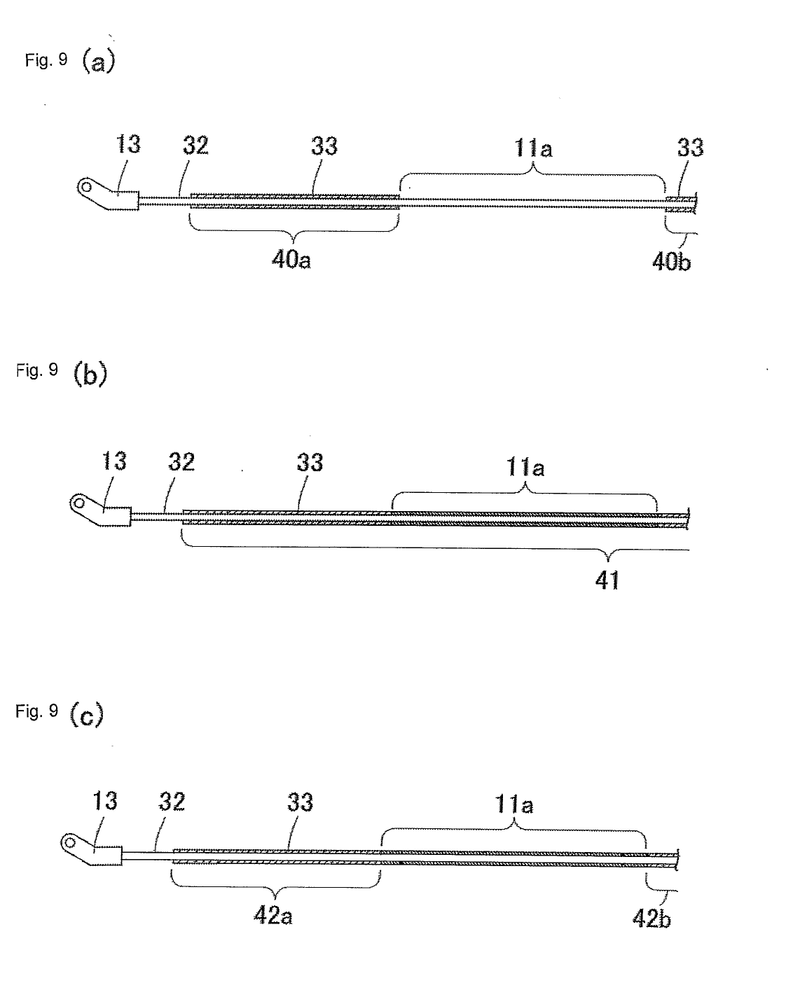

As illustrated in FIG. 9 (a), in the bendable portion 11a, the third liner member 33 also serves as the second liner member 32, and in other portions, by integrating the second line part 32 with the third liner members 33 with an adhesive agent, etc., integral portions (40a, 40b, . . . ) are formed. This can improve the transmitting performance of the rotation operation force and a bending performance of the bendable portions 11a and 11b. Note that the same applies to the bendable portion lib which is not shown in this figure.

### Modification2

As illustrated in FIG. 9 (b), by integrating the second line part 32 with the third liner members 33 in substantially the whole length with an adhesive agent, etc., an integral portion 41 is formed. In this case, the third liner member 33 preferably should have any of various slits described above in at least the bendable portion 11a. This prevents the degradation of bending performance and improves the transmitting performance of the rotation operation force greatly.
Note that the same applies to the bendable portion 11b which is not shown in this figure.
The state “integrating in substantially the whole length” contains not only a state that the second liner member is integrated with the third liner member in the whole length, but also the similar state above that the second liner member is integrated with the third liner member, in consideration of the interaction effect thereof.

### Modification3

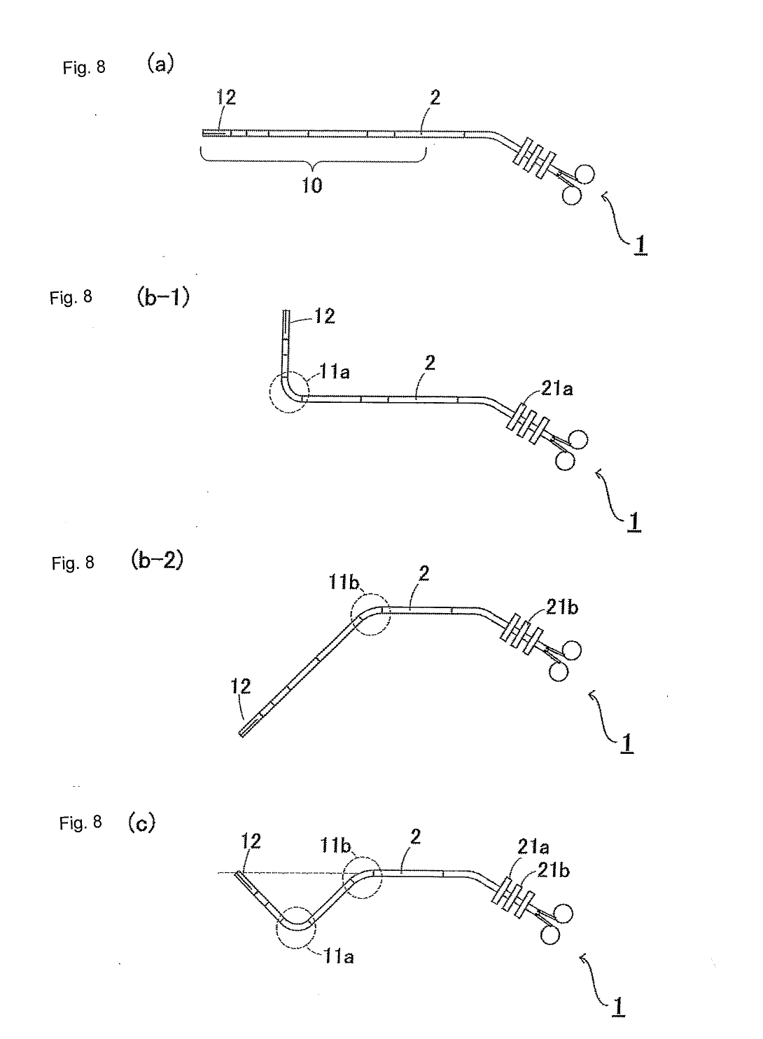

As illustrated in FIG. 9 (c), by integrating the second line part 32 with the third liner members 33 in only other parts than bendable portion 11a with an adhesive agent, etc., integral portions (42a, 42b, . . . ) may be formed. This further prevents the degradation of bending performance in the bendable portion 11a and improves the transmitting performance of the rotation operation force greatly, compared to the modification 2. Note that the same applies to the bendable portion 11b which is not shown in this figure.
FIGS. 8 (a), (b-1), (b-2) and (c) are views illustrating possible forms of the treatment tool 1 of the embodiment. The forms described above are merely examples, and possible forms of the treatment tool 1 include, but are not limited to, the forms described above
In FIG. 8 (a), the insertion part 10 is in a straight form. In this state, an operator inserts the insertion part 10 in the treatment tool 1 into a patient body through a trocar.
As illustrated in FIG. 8 (b-1), after the insertion of the insertion part 10, the operator operates the first bending operation dial 21a so that the first bendable portion 11a forms a desired bending angle, or as illustrated in FIG. 8 (b-2), the operator operates the second bending operation dial 21b so that the second bendable portion 11b forms a desired bending angle.
Subsequently, as illustrated in FIG. 8 (c), the operation of the first bending operation dial 21a or the second bending dial 21b causes the first bendable portion 11a or the second bendable portion 11b to be bent. In this case, preferably an operator should adjust the bending angle of the second bendable portion 11b so that the distal end forceps pieces 12 is on the straight line of the main body 2 of the operation part 20 side of the second bendable portion 11b, and a surgery is performed in this state. During the surgery, the operator adjusts the bending angle of the first bendable portion 11a and the second bendable portion lib, and performs treatments, such as gripping the organ of a patient or suturing it with a suture, by using a pair of the forceps pieces arranged at the tip of the pipe-like main body 2.
As described above, since the treatment tool 1 of the embodiment has two or more bendable portions 11 that can be operated to bend independently, in the insertion part to be inserted into a patient body, and its distal end forceps pieces 12 are openable/closable and rotatable, an excellent operability of the treatment tool 1 is achieved.
The treatment tool of the embodiment is used mainly for a single incision laparoscopic surgery.
In the single incision laparoscopic surgery, since an operator inserts the treatment tool into a patient body through the umbilicus and sutures the wound after the surgery, this procedure causes very little scarring. The patient recovery is also fast compared to the conventional endoscopic surgery, and it is not uncommon that the patient can leave the hospital within several days after the surgery. The single incision laparoscopic surgery is an excellent surgical procedure minimizing the burdens and the scarrings on patients.
Concerning to the endoscopic surgery, however, the single incision laparoscopic surgery has problems. Namely, in addition to the problem that an operator must remote-control an endoscope or a treatment tool without direct-viewing the affected area, it is also difficult to perform a surgery by using the endoscope or the treatment tool being inserted into one trocar. Therefore training of physicians has become an important issue because the operators are required to acquire the advanced technology.
On the other hand, the operability of the treatment tool is an important element to determine the success or failure of the surgery, and damages to an organ differ depending on whether the operator can grip the organ at an appropriate angle and with an appropriate force. The prolonged surgery due to the operability failure of the treatment tool will impair the strength of the patient and reduce the success rate of the surgery greatly. It should not be ignored how the surgery under a tense situation will affect the operator psychologically. However, as described above, the treatment tools for an endoscopic surgery including the single incision laparoscopic surgery is subjected to severe structural constraints due to the inside diameter of the trocar, and as a result it is not easy to provide the treatment tools with a high operability.
Therefore, it is certain that development of the treatment tool with a high operability of the present invention increases the number of the operators who can perform a single incision laparoscopic surgery appropriately, improves the success rate and the quality of surgeries and remarkably contributes to the prevalence of the single incision laparoscopic surgeries.
In addition, technical ideas of the present invention can be broadly applied to not only treatment tools for the single incision laparoscopic surgeries but also other medical tools.

### REFERENCE SIGNS LIST

1 treatment tool
2 main body
10 insertion part
11 bendable portion
11a first bendable portion
11b second bendable portion
12 forceps pieces
13 link mechanism
14 locking member
20 operation part
21 bending operation part
21a first bending operation part
21b second bending operation part
22 open-close operation part
23 rotation operation part
24 link mechanism
25 bending portion
26 locking member
31 liner member (for bend-drive)
32 liner member (for open-close-drive)
33 liner member (for rotate-drive)
34 pipe
35 hollow portion
36 slit
37 non-slit portion
38 slit forming part
39 slit forming part
40 integral portion
41 integral portion
42 integral portion

1. A treatment tool for an endoscopic surgery comprising:
an insertion part disposed at one end of a pipe-like main body; and an operation part disposed at the other end of the main body,
wherein the insertion part has two or more bendable portions and a pair of forceps pieces,
the operation part has bending operation parts corresponding to each of the bendable portions, an open-close operation part and a rotation operation part, the bendable portions can be operated to bend by operating first linear members connected to the bending operation parts to move back and forth in axial direction inside the main body,
the forceps pieces can be operated to open and close by operating a second linear member connected to the open-close operation part to move back and forth in axial direction inside the main body, and
the forceps pieces can be operated to rotate by operating a third liner member connected to the rotation operation part to rotate around the axis inside the main body.

2. The treatment tool according to claim 1,
wherein the third liner member is a pipe having one spirally formed slit at least in the bendable portions.

3. The treatment tool according to claim 1,
wherein the third liner member is a pipe having a plurality of spirally formed parallel slits at least in the bendable portions.

4. The treatment tool according to claim 3,
wherein the each slit has at least one non-slit portion.

5. The treatment tool according to claim 1,
wherein the third liner member is a pipe which has a plurality of slits parallel to axial direction , and
the slits are formed in at least a part of an outer circumference circle vertical to an axis of the pipe, at least in the bendable portions.

6. The treatment tool according to claim 1,
wherein the third liner member is made of a p titanium alloy.

7. The treatment tool according to claim 1,
wherein at least in any of the bendable portions, the third liner member also serves as the second liner member.

8. The treatment tool according to claim 1,
wherein the second line part is integrated with the third liner members, at least partially to the length direction.

9. The treatment tool according to claim 8,
wherein the second liner member is integrated with the third liner member in the whole length.

10. The treatment tool according to claim 8,
wherein the second liner member is integrated with the third liner member in other parts than the bendable portions.
